SEQUENCE LISTING (6 sequences)

SEQ ID NO: 1 - DNA, 816 bp, Ambrosiozyma monospora
atgactgactacattccaacttttagattcgatggccacttaaccattgtcacaggtgcctgtggtggtttagctgaagctttaatcaagggtttgttggcctacggttctgacattgctttgcttgatatcgaccaagaaaagactgctgccaaacaagccgaataccacaaatacgctactgaagaattgaagttgaaagaagttccaaagatgggttcatatgcctgtgatatttctgattctgataccgttcacaaggtgtttgctcaagttgctaaggattttggtaagttgccattgcacttggttaacacagctggttactgtgaaaacttcccatgtgaagattacccagccaagaacgctgagaagatggtgaaggttaacttgttgggttctttgtatgtttctcaagcctttgctaagccattgatcaaagaaggtatcaagggtgcttctgttgttttgattggttctatgtctggtgccattgtcaacgatcctcaaaaccaagttgtctacaacatgtccaaggctggtgttatccatttggctaagactttggcttgtgaatgggctaagtacaacatcagagttaattctttaaacccaggttacatctacggtcctttgaccaagaatgttatcaatggtaacgaagaattgtacaacagatggatctctggtatcccacaacaaagaatgtccgaaccaaaggaatacattggtgctgttttgtacttgctttctgaatctgctgcttcatacactactggtgccagcttactggttgatggtggtttcacttcttgg

SEQ ID NO: 2 - protein, 272 aa, Ambrosiozyma monospora
MTDYIPTFRFDGHLTIVTGACGGLAEALIKGLLAYGSDIALLDIDQEKTAAKQAEYHKYATEELKLKEVPKMGSYACDISDSDTVHKVFAQVAKDFGKLPLHLVNTAGYCENFPCEDYPAKNAEKMVKVNLLGSLYVSQAFAKPLIKEGIKGASVVLIGSMSGAIVNDPQNQVVYNMSKAGVIHLAKTLACEWAKYNIRVNSLNPGYIYGPLTKNVINGNEELYNRWISGIPQQRMSEPKEYIGAVLYLLSESAASYTTGASLLVDGGFTSW

SEQ ID NO: 3 - DNA, 21 bp, Artificial Sequence
  Primer
tataacgcgtttggaatcact

SEQ ID NO: 4 - DNA, 21 bp, Artificial Sequence
  Primer
taaatttctggcaaggtagac

SEQ ID NO: 5 - DNA, 54 bp, Artificial Sequence
  Primer
gactggatccatcatgcatcatcatcatcatcatatgactgactacattccaac

SEQ ID NO: 6 - DNA, 32 bp, Artificial Sequence
  Primer
atgcggatccctatatataccggaaaatcgac

(19) United States
(12) Patent Application Publication
(10) Pub. No.: US 2004/0132074 A1
(43) Pub. Date: Jul. 8, 2004

(54) New enzyme for an in vivo and in vitro utilisation of carbohydrates

(75) Inventors: Ritva Verho, Espoo (FI); Peter Richard, Helsinki (FI); Merja Penttila, Helsinki (FI)

Correspondence Address: ROTHWELL, FIGG, ERNST & MANBECK, P.C., 1425 K STREET, N.W., SUITE 800, WASHINGTON, DC, 20005, US

(73) Assignee: VALTION TEKNILLINEN TUTKIMUSKESKUS

(21) Appl. No.: 10/720,018

(22) Filed: Nov. 24, 2003

Related U.S. Application Data
(63) Continuation-in-part of application No. 10/257,821, filed on Mar. 10, 2003.
Application No. 10/257,821 is a 371 of international application No. PCT/FI02/00125, filed on Feb. 15, 2002.

(30) Foreign Application Priority Data
Feb. 16, 2001 (FI) 20010308
Sep. 12, 2003 (FI) 20031307

(51) Int. Cl. C12Q 1/68; C07H 21/04; C12N 9/02; C12N 1/18

(52) U.S. Cl. 435/6; 435/69.1; 435/189; 435/254.2; 435/320.1; 536/23.2

(57) ABSTRACT

An isolated DNA molecule includes a gene encoding an enzyme protein which has an NADH dependent L-xylulose reductase activity. The isolated DNA molecular may be included in a vector, and a genetically modified microorganism transformed by such vector. The genetically modified microorganisms are utilized to produce fermentation products.

### CROSS-REFERENCE TO RELATED APPLICATION

This application is a continuation-in-part of application Ser. No. 10/257,821, filed Mar. 10, 2003, which is the National Stage of International Application No. PCT/FI02/00125, filed Feb. 15, 2002.

### FIELD OF THE INVENTION

The present invention relates to an isolated DNA molecule comprising a gene encoding an enzyme which can be used for an in vivo and in vitro utilisation of carbohydrates, such as sugars or their derivatives, as well as to a microorganism transformed with said DNA molecule. The invention is further directed to the enzyme protein encoded by said DNA molecule and to the use thereof for the conversion of sugars or their derivatives.

### BACKGROUND OF THE INVENTION

Biological waste material from industry including agriculture contains e.g. carbohydrates, such as sugars. The conversion of such waste to useful products has been of interest and challenge i.e. in the field of biotechnology for a long time.

As a specific example of carbohydrates the sugar L-arabinose can be mentioned, which is a major constituent of plant material. L-arabinose fermentation is therefore also of potential biotechnological interest.

Fungi that can use L-arabinose are not necessarily good for industrial use. Many pentose utilising yeast species for example have a low ethanol tolerance, which makes them unsuitable for ethanol production. One approach would be to improve the industrial properties of these organisms. Another is to give a suitable organism the ability to use L-arabinose.

For the catabolism of L-arabinose two distinctly different pathways are known, a bacterial pathway and a fungal pathway (see FIG. 1). In the bacterial pathway the three enzymes L-arabinose isomerase, ribulokinase and L-ribulose-5-phosphate 4-epimerase convert L-arabinose to D-xylulose 5-phosphate. The fungal pathway was first described by Chiang and Knight: “A new pathway of pentose metabolism” in Biochem Biophys Res Commun, 3, 1960, 554-559, for the mould Penicillium chrysogenum. It also converts L-arabinose to D-xylulose 5-phosphate but through the enzymes L-arabinose reductase, L-arabinitol 4-dehydrogenase, L-xylulose reductase, xylitol dehydrogenase and xylulokinase. In this pathway the L-arabinose reductase and the L-xylulose reductase use NADPH as a cofactor, while L-arabinitol 4-dehydrogenase and xylitol dehydrogenase use NAD⁺ as a cofactor.

The same pathway was described for the mould Aspergillus niger (Witteveen et al.: “L-arabinose and D-xylose catabolism in Aspergillus niger” in J Gen Microbiol, 135, 1989, 2163-2171). The pathway was expressed in Saccharomyces cerevisiae using genes from the mould Hypocrea jecorina and shown to be functional, i.e. the resulting strain could grow on and ferment L-arabinose, however at very low rates (Richard et al.: “Cloning and expression of a fungal L-arabinitol 4-dehydrogenase gene” in J Biol Chem, 276, 2001, 40631-7; Richard et al.: “The missing link in the fungal L-arabinose catabolic pathway, identification of the L-xylulose reductase gene” in Biochemistry, 41, 2002, 6432-7; Richard et al.: “Production of ethanol from L-arabinose by Saccharomyces cerevisiae containing a fungal L-arabinose pathway” in FEMs Yeast Res, 3, 2003, 185-9). Information about the corresponding pathway in yeast is rare. Shi et al.: “Characterization and complementation of a Pichia stipitis mutant unable to grow on D-xylose or L-arabinose” in Appl Biochem Biotechnol, 84-86, 2000, 201-16, provided evidence that the yeast pathway requires a xylitol dehydrogenase. In a mutant of Pichia stipitis, which was unable to grow on L-arabinose, overexpression of a xylitol dehydrogenase could restore growth on L-arabinose.

Dien et al.: “Screening for L-arabinose fermenting yeasts” in Appl Biochem Biotechnol, 57-58, 1996, 233-42, tested more than 100 yeast species for L-arabinose fermentation. Most of them produced arabinitol and xylitol indicating that the yeast pathway is similar to the pathway of moulds and not to the pathway of bacteria. However little is known about the cofactor specificities of the catalytic steps in a yeast pathway.

The fungal L-arabinose pathway has similarities to the fungal D-xylose pathway. In both pathways the pentose sugar goes through reduction and oxidation reactions where the reductions are NADPH-linked and the oxidations NAD⁺-linked. D-xylose goes through one pair of reduction and oxidation reaction and L-arabinose goes through two pairs. The process is redox neutral but different redox cofactors, i.e. NADPH and NAD⁺ are used, which have to be separately regenerated in other metabolic pathways. In the D-xylose pathway an NADPH-linked reductase converts D-xylose into xyhtol, which is then converted to D-xylulose by an NAD⁺-linked dehydrogenase and to D-xylulose 5-phosphate by xylulokinase. The enzymes of the D-xylose pathway can all be used in the L-arabinose pathway. The first enzyme in both pathways is an aldose reductase (EC 1.1.1.21). The enzymes have been characterised in different fungi and the corresponding genes cloned. The Pichia stipitis enzyme is special as it can use NADPH and NADH as a cofactor (Verduyn et al.: “Properties of the NAD(P)H-dependent xylose reductase from the xylose-fermenting yeast Pichia stipitis” in Biochem J, 226, 1985, 669-77). It is also unspecific towards the sugar and can use either L-arabinose or D-xylose with approximately the same rate to produce L-arabinitol or xylitol respectively. Also the xylitol dehydrogenase, which is also known as D-xylulose reductase EC 1.1.1.9, and xylulokinase EC 2.7.1.17 are the same in the D-xylose and L-arabinose pathway of fungi. Genes for the D-xylulose reductase and xylulokinase are known from various fungi. Genes coding for L-arabinitol 4-dehydrogenase (EC 1.1.1.12) or L-xylulose reductase (EC 1.1.1.10) have recently been described in the patent application WO 02/066616.

The catabolism of L-arabinose using the fungal pathway is slow. It is believed that this is due to the use of different cofactors in the pathway. For the conversion of one mole L-arabinose two moles of NADPH and two moles of NAD⁺ are converted to NADP⁺ and NADH respectively, i.e. although the overall reaction in the pathway is redox neutral, an imbalance of redox cofactors is generated. This could be circumvented if the pathway would only use the NAD⁺/NADH cofactor couple.

L-xylulose reductases are described for moulds and higher animals. From hamster liver a gene was identified, which coded for diacetyl reductase that had also L-xylulose reductase activity (Ishikura et al.: “Molecular cloning, expression and tissue distribution of hamster diacetyl reductase. Identity with L-xylulose reductase” in Chem Biol Interact, 130-132, 2001, 879-89).

All these L-xylulose reductase activities have in common, that they are strictly coupled to NADPH. To our knowledge there is no report about an L-xylulose reductase activity that is coupled to NADH.

Hallborn et al.: “A short-chain dehydrogenase gene from Pichia stipitis having D-arabinitol dehydrogenase activity” in Yeast, 11, 1995, 839-47, described an NAD⁺ dependent D-arabinitol dehydrogenase, which is forming D-ribulose from D-arabinitol. In their report they also mention activity with NAD⁺ and xylitol, however it is was concluded that D-xylulose is the product of this activity.

There exists a continuous need for providing industrially applicable biotechnological means for the conversion of cheap biomass to useful products.

### SUMMARY OF THE INVENTION

Accordingly, the present invention provides a new isolated DNA molecule that contains a gene encoding an enzyme protein that exhibits preferable properties.

Further, the invention provides a genetically engineered DNA molecule comprising the gene of the invention, which enables the transforming and expression of the gene of the invention conveniently in a host microorganism.

The invention further provides a genetically modified microorganism, which is transformed with the DNA molecule of the invention and is capable for effectively fermenting carbohydrates, such as sugars or their derivatives, from a biomaterial to obtain useful fermentation products.

Another aim of the invention is to provide an enzyme protein which can be expressed by a host for the conversion of carbohydrates, particularly sugars or their derivatives, such as sugar alcohols, to useful conversion products in a fermentation medium, or which is in the form of an enzymatic preparation for in vitro conversion of the above mentioned carbohydrates to useful end products or intermediate products.

### BRIEF DESCRIPTION OF THE DRAWINGS

FIG. 1. The fungal and the bacterial pathway for L-arabinose utilisation.

FIG. 2. The cDNA sequence of SEQ ID No. 1 comprised in a DNA molecule encoding an NADH dependent L-xylulose reductase as well as the amino acid sequence of SEQ ID No.2 encoded by said cDNA.

### DETAILED DESCRIPTION OF THE INVENTION

The present invention provides for the first time an isolated DNA molecule, which comprises a gene encoding an enzyme protein, which exhibits an NADH dependent L-xylulose reductase activity. The isolation and the identification procedure are described below.

The term “an NADH dependent L-xylulose reductase” or “an enzyme protein which has an NADH dependent L-xylulose reductase activity” means herein that, the enzyme protein of the present invention exhibits L-xylulose reductase activity and uses NADH as the cofactor, i.e. is strictly NADH dependent enzyme, which is contrary to the known L-xylulose reductases which use merely NADPH as the cofactor.

The term “gene” means herein a nucleic acid segment which comprises a nucleic acid sequence encoding an amino acid sequence characteristic of a specific enzyme protein. Thus the gene of the invention comprises a nucleic acid sequence encoding the amino acid sequence characteristic of an enzyme protein which has the NADH dependent L-xylulose reductase activity. The “gene” may optionally comprise further nucleic acid sequences, e.g. regulatory sequences.

It is evident that the terms “DNA molecule”, “DNA sequence” and “nucleic acid sequence” include cDNA (complementary DNA) as well.

Due to the NADH dependency, the present L-xylulose reductase enzyme of the invention thus provides an alternative for the redox cofactor regeneration in metabolic pathways encompassing L-xylulose reductase as one of the enzymes of the pathway. Particularly, the present L-xylulose reductase improves the NADP+-NAD+ balance e.g. in a fungal L-arabinose pathway. As a result, an industrially beneficial fungal pathway, e.g. L-arabinose pathway, can be provided, which can convert L-arabinose to D-xylulose without generating an imbalance of redox cofactors.

Preferably, the gene of the DNA molecule of the invention encodes an NADH dependent L-xylulose reductase which exhibits a catalytic activity for the reversible conversion of a sugar to a sugar alcohol with the sugar having the keto group at the carbon 2, C2, and the sugar alcohol having the hydroxyl group of the C2 in L-configuration in a Fischer projection. Particularly, said NADH dependent L-xylulose reductase exhibits a catalytic activity for the reversible conversion of L-xylulose to xylitol. Another useful activity is the reversible reaction of D-xylulose and D-ribulose to D-arabinitol.

In one preferable embodiment of the invention the gene of the DNA molecule encodes an enzyme protein which comprises the amino acid sequence of SEQ ID NO. 2 or a functionally equivalent variant thereof.

In another preferable embodiment of the invention the isolated DNA molecule comprises a gene coding for NADH dependent L-xylulose reductase of fungal origin, i.e. the gene sequence has the sequence obtainable from a fungal L-xylulose reductase, or an equivalent gene sequence thereof. A preferred example of the fungal origin is Ambrosiozyma monospora, particularly the above-mentioned strain NRRL Y-1484.

According to a further preferable embodiment, the gene of the DNA molecule comprises the nucleic acid sequence of SEQ ID No. 1 or a functionally equivalent variant thereof.

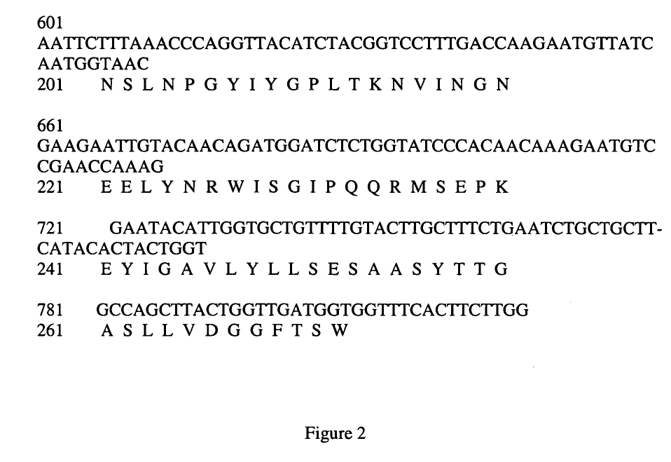

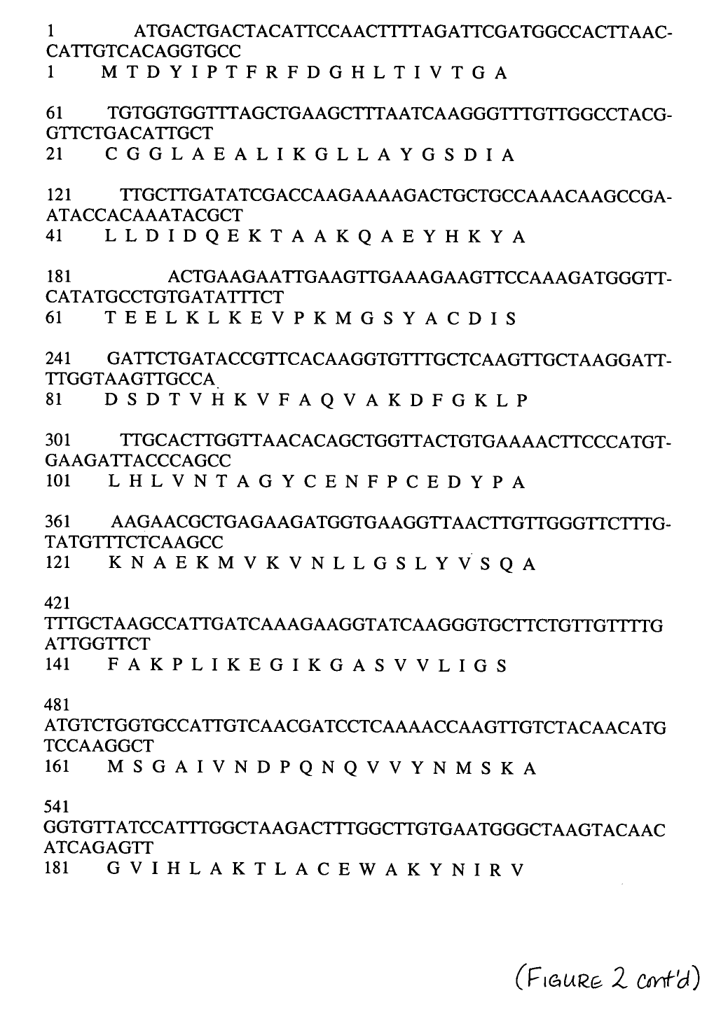

A deposit has been made for the cDNA sequence of SEQ ID No. 1 by VTT Biotechnology, address: P.O. Box 1500, Tietotie 2, 02044 VTT, Finland, in the International Depositary Authority, Deutsche Sammlung von Mikroorganismen und Zellkulturen GmbH (DSMZ, Mascheroder Weg 1b, D-38124 Braunschweig), under the terms of Budapest Treaty, on Aug. 5, 2003 (5.8.2003), and have been assigned Accession Number DSM 15821. The deposited strain S. cerevisiae, DSM 15821, comprises the cDNA of SEQ ID No. 1 (see also FIG. 2), which has been referred in the experimental part below also as ALX1 gene, on a multicopy plasmid under a constitutive yeast promoter. In this strain the L-xylulose reductase is expressed. The deposited nucleic acid sequence originates from a known Ambrosiozyma monospora NRRL Y-1484. More details of the nucleic acid and amino acid sequence of the invention, plasmid used in the deposited strain and the deposited strain are given in the experimental part below, e.g. in Examples 1 and 2, and in FIG. 2. Also the sequence listing of SEQ ID NO.1 and SEQ ID NO.2 are included to support this data.

It is well known that genes from different organisms encoding enzymes with the same catalytic activity have sequence similarities and these similarities can be exploited in many ways by those skilled in the art to clone other genes from other organisms with the same catalytic activity. Such genes are also suitable to practise the present invention.

It is thus evident that many small variations in the nucleotide sequence of a gene do not significantly change the catalytic properties of the encoded protein. For example, many changes in nucleotide sequence do not change the amino acid sequence of the encoded protein. Also an amino acid sequence can have variations which do not change the functional properties of a protein, in particular they do not prevent an enzyme from carrying out its catalytic function. Such variations in the nucleotide sequence of DNA molecules or in an amino acid sequence are known as “functionally equivalent variants”, because they do not significantly change the function of the gene to encode a protein with a particular function, e.g. catalysing a particular reaction or, respectively, of the protein with a particular function. Thus such functionally equivalent variants, including fragments, of the nucleotide sequence of SEQ ID NO 1 and, respectively, of the amino acid sequence of SEQ ID NO 2, are encompassed within the scope of the invention.

Furthermore, the invention is also directed to a genetically engineered DNA molecule, i.e. a recombinant DNA, suitably to a vector, especially to an expression vector, which comprises the gene of the DNA molecule of the invention as defined above so that it can be expressed in a host cell, i.e. a microorganism. In the recombinant DNA, the gene of the invention may i.a. be operably linked to a promoter. The vector can be e.g. a conventional vector, such as a virus, e.g. a bacteriophage, or a plasmid, preferably a plasmid. The construction of an expression vector is within the skills of an artisan. The general procedure and specific examples are described below.

Moreover, the DNA molecule as defined above is preferably used for transforming a microorganism for producing the NADH dependent L-xylulose enzyme comprising an amino acid encoded by the gene of the DNA molecule as defined above. Accordingly, a genetically modified microorganism that comprises the DNA molecule of the invention as defined above for the expression of said NADH dependent L-xylulose is provided.

The DNA molecule of the invention can be transferred to any microorganism suitable for the production of the desired conversion products from a biomaterial that comprises carbohydrates, preferably sugars or sugar derivatives. It would be evident for a skilled person that “a suitable microorganism” means: (1) it is capable of expressing the gene of the DNA of the invention encoding said enzyme protein and, optionally, (2) it can produce further enzymes that are needed for an industrial conversion of the raw material, i.e. biomaterial, to obtain the desired products, as well as (3) it can tolerate the formed conversion products, i.e. any intermediates and/or the end product(s), to enable the industrial production. The transformation (or transfection) of the microorganism can be effected in a manner known in the field of biotechnology, preferably by using the vector of the invention as described above or as described in the example part below.

Naturally, either the biomaterial to be utilised by said microorganism of the invention comprises the sugar product that is convertible by the present NADH dependent L-xylulose reductase, or the microorganism is capable to express further genes to produce enzymes that are needed for the conversion of the starting biomaterial to a sugar product utilisable by said reductase expressed by the gene of the invention.

Furthermore, depending on the desired conversion product, the microorganism may comprise additional genes for the expression of one or more further enzymes that can convert the conversion product of the present NADH dependent L-xylulose reductase enzyme to the desired product. Preferably, the enzyme of the invention and at least part of said optional further enzyme(s) are members of the same metabolic pathway. Moreover, the microorganism of the invention may comprise genes for the enzymes of two or more metabolic pathways so that the product of one of the pathways can be utilised by another metabolic pathway.

It is also evident that the said optional further gene(s) needed e.g. for expressing the enzymes of the metabolic pathway of the enzyme product of the invention and/or of further pathways may be contained in the genome of the microorganism, or the microorganism may be transformed with any lacking gene of said further gene(s).

The genetically modified microorganism of the invention has an ability to utilise a carbohydrate, such as a sugar or a derivative thereof, such as a sugar alcohol. The invention provides a method for producing fermentation product(s) from a carbon source which comprises a carbohydrate, such as a sugar or a derivative thereof, including a step of culturing the genetically modified microorganism as defined above in the presence of the carbon source in suitable fermentation conditions and, optionally, recovering the desired fermentation product(s).

In one preferred embodiment of the invention the genetically modified microorganism has an increased ability to utilise L-arabinose. Preferably, said microorganism produces product(s) of the fungal L-arabinose pathway and/or of the pentose phosphate pathway. Particularly, the genetically modified microorganism utilises biomaterial that comprises L-arabinose and contains at least the genes of the fungal L-arabinose pathway, which encode the enzymes of aldose reductase, especially EC 1.1.1.21, and of L-arabinitol 4-dehydrogenase, especially EC 1.1.1.12, for the expression thereof. More particularly, said microorganism further contains genes of the fungal L-arabinose pathway, which encode the enzymes of D-xylulose reductase, especially EC 1.1.1.9 and/or xylulokinase, especially EC 2.7.1.17, and, optionally, genes encoding for the enzymes of the known pentose phosphate pathway.

The desirable conversion products obtainable by the genetically modified microorganism may include the conversion products of the fungal L-arabinose pathway, i.a. L-arabinitol, L-xylulose, xylitol, D-xylulose and/or D-xylulose 5-phosphate; and the conversion products of the known pentose phosphate pathway or other pathways that can utilise e.g. the end conversion product D-xylulose 5-phosphate of the fungal L-arabinose pathway, i.a. ethanol and/or lactic acid.

A genetically modified microorganism of the invention is preferably a fungus, which can be selected from a yeast and a filamentous fungus. Suitably the fungus is a yeast.

Industrial yeasts have process advantages such as high ethanol tolerance, tolerance of other industrial stresses and rapid fermentation. They are normally polyploid and their genetic engineering is more difficult compared to laboratory strains, but methods for their engineering are known in the art (see, e.g., Blomqvist et al: “Chromosomal integration and expression of two bacterial α-acetolactate decarboxylase genes in brewer's yeast” in Appl. Environ. Microbiol. 57, 1991, 2796-2803; Henderson et al: “The transformation of brewing yeasts with a plasmid containing a gene for copper resistance” in Current Genetics, 9, 1985, 133-138). Yeasts, which may be transformed according to the present invention for the utilisation of a carbon source of the invention, e.g. L-arabinose, include i.a. a strain of Saccharomyces species, Schizosaccharomyces species, e.g. Schizosaccharomyces pombe, Kluyveromyces species, Pichia species, Candida species or Pachysolen species. Also Schwanniomyces spp., Arxula, spp., Trichosporon spp., Hansenula spp. and Yarrowia spp. could be mentioned. One preferable yeast is e.g. an industrial strain of S. cerevisiae, e.g. a brewer's, distiller's or baker's yeast.

Furthermore, also a filamentous fungus can be transformed according to the present invention. Such fungi includes i.a. a strain of Trichoderma species, Neurospora species, Fusarium species, Penicillium species, Humicola species, Tolypocladium geodes, Trichoderma reesei (Hypocrea jecorina), Mucor species, Trichoderma longibrachiatum, Aspergillus nidulans, Aspergillus niger or Aspergillus awamori.

Preferably the transformed microorganism of the invention is an industrial strain of S. cerevisiae which comprises the transformed gene of the invention and additionally the further genes of the fungal L-arabinose pathway and optionally pentose phosphate pathway, and which can convert a carbon source comprising at least one of the utilisable products of the L-arabinose pathway, preferably L-arabinose, to the end product and/or intermediate product(s) of said pathway, or, optionally to product(s) of the pentose phosphate pathway. All or part of said further genes may be present in the genome of the strain or the strain may be a genetically engineered strain, which has been transformed with all or part of said further genes. A suitable example is S. cerevisiae which is transformed according to the present invention and produces ethanol from a starting biomaterial.

The invention is not restricted to yeasts and other fungi. The genes encoding L-xylulose reductase can be expressed in any organism such as bacteria, plants or higher eukaryotes unable to use or inefficient in using L-arabinose by applying the genetic tools suitable and known in the art for that particular organism.

A new enzyme protein, which has an NADH dependent L-xylulose reductase activity, has also now been isolated and identified.

As a further aspect of the invention also an enzyme protein is provided, which has an NADH dependent L-xylulose reductase activity and comprises an amino acid sequence encoded by the gene of the DNA molecule as defined above.

In a specific embodiment of the invention the enzyme protein comprises the amino acid sequence of SEQ ID NO. 2 or a functionally equivalent variant thereof. The functionally equivalent variants include an amino acid sequence having at least 30%, preferably at least 50%, suitably at least 70%, e.g. at least 90% sequence identity to SEQ ID NO.2.

The invention is further directed to an in vitro enzymatic preparation, which contains at least the enzyme protein as defined above. The preparation may be in the form known in the field of enzyme preparations, e.g. in a pulverous such as freeze-dried form or in a solution. The pulverous form of the preparation may be used as such or dissolved in a suitable solution before the use. Similarly as above for the genetically modified microorganism, the enzyme preparation of the invention may contain one or more further enzymes, which can convert the starting material to a sugar product utilisable by the enzyme product of the invention and/or convert the resulted conversion product of the present enzyme to further conversion products. The convertible raw materials, the further enzymes and/or the desired end products may be e.g. as defined above for said transformed microorganism.

Moreover, the invention provides the use of an NADH dependent L-xylulose reductase enzyme as defined above for the conversion of a sugar with a keto group in C2 position to a sugar alcohol wherein hydroxyl group of C2 is in L-configuration in the Fischer projection, or for the reversed conversion thereof, preferably for the conversion of L-xylulose to xylitol, or for the reversed conversion thereof.

In one embodiment of the conversion method the enzyme is produced by the genetically engineered microorganism as defined above in a fermentation medium which comprises the sugar or, respectively, the sugar alcohol, in fermentation conditions that enable the conversion by the produced enzyme.

In a further embodiment, the conversion method is carried out as an in vitro conversion using the enzyme preparation as defined above. Such preparation can be obtained by expressing the enzyme in a microorganism and recovering the obtained enzyme product, or by chemically preparing the enzyme product e.g. in a manner known from the peptide chemistry. The conversion products of the enzyme preparation can be used as such (end products) or as intermediate products that are further converted e.g. by biotechnological or chemical means.

Description of the Procedures for Isolating and Identifying the DNA Molecule of the Invention

To identify the gene for the L-xylulose reductase of the invention different approaches are possible and a person knowledgeable in the art might use different approaches. One approach is to purify the protein with the corresponding activity and use information about this protein to clone the corresponding gene. This can include the proteolytic digestion of the purified protein, amino acid sequencing of the proteolytic digests and cloning a part of the gene by PCR with primers derived from the amino acid sequence. The rest of the DNA sequence can then be obtained in various ways. One way is from a cDNA library by PCR using primers from the library vector and the known part of the gene. Once the complete sequence is known the gene can be amplified from the cDNA library and cloned into an expression vector and expressed in a heterologous host. This is a useful strategy if screening strategies or strategies based on homology between sequences are not suitable.

Another approach to clone a gene is to screen a DNA library. This is especially a good and fast procedure, when overexpression of a single gene causes a phenotype that is easy to detect. Now that we have disclosed that transformation of a xylose-utilising fungus with genes encoding L-arabinitol dehydrogenase and L-xylulose reductase confers the ability to grow in L-arabinose, another strategy to find the genes for L-xylulose reductase is the following: A strain with all the gene of the L-arabinose pathway except the L-xylulose reductase can be constructed, transformed with a DNA library, and screened for growth on L-arabinose.

There are other ways and possibilities to clone a gene for an L-xylulose reductase:

One could screen for example for growth on L-xylulose to find the L-xylulose reductase.

One can screen existing databanks for genes with homology to genes from related protein families and test whether they encode the desired enzyme activity. Now that we have disclosed sequence for a gene L-xylulose reductase (SEQ ID NO 1), it is easy for a person skilled in the art to screen data banks for genes homologous to SEQ ID NO 1. Homologous genes can also be readily found by physical screening of DNA libraries using probes based on SEQ ID NO 1. Suitable DNA libraries include libraries generated from DNA or RNA isolated from fungi and other microbes able to utilise L-arabinose or L-xylulose.

For a person skilled in the art there are different ways to identify the gene, which codes for a protein with the desired enzyme activity. The methods described here illustrate our invention, but any other method known in the art may be used.

All or part of the genes for the L-arabinose pathway including the present NADH dependent L-xylulose reductase can be introduced to a new host organism, which is lacking this pathway or has already part of the pathway. For example a fungus that can utilise D-xylose might only require the enzymes that convert L-arabinitol to xylitol. Expression of L-arabinitol 4-dehydrogenase and L-xylulose reductase would then be sufficient to complete the L-arabinose pathway. Enzyme assays have been described for all the steps of the fungal arabinose pathway (Witteveen et al., 1989) and these can be used if necessary to help identify the missing or inefficient steps in a particular host.

In the examples the PGK1 promoter from S. cerevisiae was used for the expression of L-xylulose reductase. The promoter is considered strong and constitutive. Other promoters, which are stronger or less strong, can be used. It is also not necessary to use a constitutive promoter. Inducible or repressible promoters can be used, and may have advantages, for example if a sequential fermentation of different sugars is desired.

In our example we used a plasmid for the gene L-xylulose reductase. The plasmid contained a selection marker. The genes can also be expressed from a plasmid without a selection marker or can be integrated into the chromosomes. The selection marker was used to find successful transformations more easily and to stabilise the genetic construct. The yeast strain was transformed with the lithium acetate procedure. Other transformation procedures are known in the art, some being preferred for a particular host, and they can be used to achieve our invention.

Specific Embodiments of the Invention

According to one preferable embodiment of the invention, the inability of a fungus to utilize L-arabinose efficiently is solved by a genetic modification of the fungus, which is characterised in that the fungus is transformed with a gene for an NADH dependent L-xylulose reductase.

According to another embodiment a microorganism, preferably a fungus, is transformed with all or some of the genes coding for the enzymes of the L-arabinose pathway, i.e. at least with aldose reductase, L-arabinitol 4-dehydrogenase and the present L-xylulose reductase, and optionally with D-xylulose reductase and/or xylulokinase. Preferably, the microorganism is transformed with all the genes of the L-arabinose pathway. The resulting microorganism, e.g. the fungus is then able to utilise L-arabinose more efficiently.

In a further embodiment, a fungus, such as a genetically engineered S. cerevisiae, that can use D-xylose but not L-arabinose is transformed with genes for L-arabinitol 4-dehydrogenase and L-xylulose reductase for utilising L-arabinose.

By the term “utilisation” is meant here that the organism can use a carbohydrate, e.g. a sugar or a derivative thereof, such as L-arabinose, as a carbon source or as an energy source or that it can convert said product, e.g. L-arabinose, into another compound that is a useful substance.

The invention is described below with a preferred embodiment in order to show in practice that a fungal microorganism can be genetically engineered to utilise a biomaterial comprising carbohydrates, such as sugars or derivatives thereof, such as L-arabinose. Some fungi can naturally utilise e.g. L-arabinose, others cannot. It can be desirable to transfer the capacity of utilising L-arabinose to a organism lacking the capacity of L-arabinose utilisation but with other desired features, such as the ability to tolerate industrial conditions or to produce particular useful products, such as ethanol or lactic acid or xylitol. In order to transfer the capacity of L-arabinose utilisation by means of genetic engineering it is essential to know all the genes of a set of enzymes that can function together in a host cell to convert L-arabinose into a derivative, e.g. D-xylulose 5-phosphate, that the host can catabolise and so produce useful products. This set of enzymes can then be completed in a particular host by transforming that host with the gene or genes encoding the missing enzyme or enzymes.

One example is to genetically engineer S. cerevisiae to utilise L-arabinose. S. cerevisiae is a good ethanol producer but lacks the capacity for L-arabinose utilisation. Other examples are organisms with a useful feature but lacking at least part of a functional L-arabinose pathway.

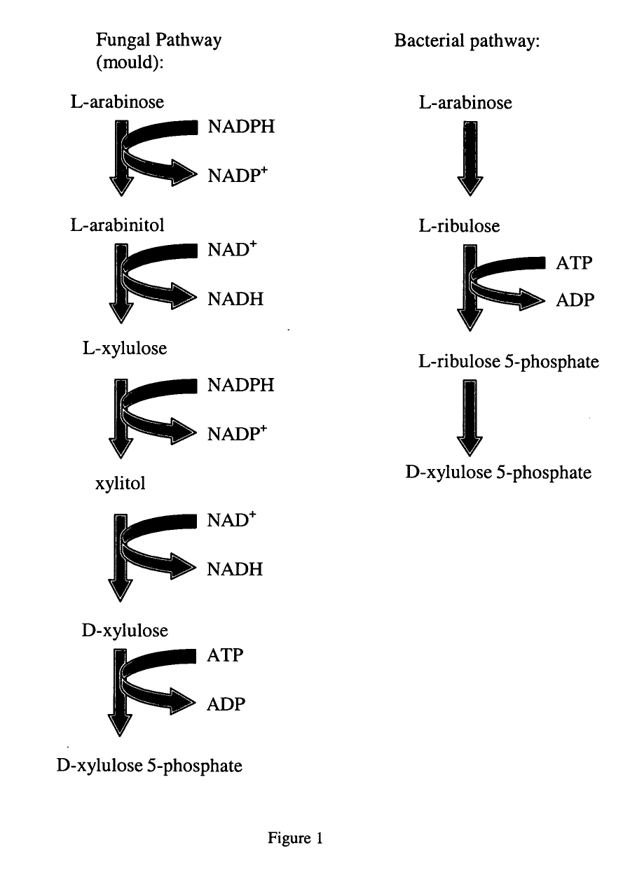

An L-arabinose pathway believed to function in fungi is shown in the FIG. 1. Genes coding for the aldose reductase (EC 1.1.1.21), the D-xylulose reductase (EC 1.1.1.9) and xylulokinase (EC 2.7.1.17) are known. Also the two additional genes required, i.e. genes for L-arabinitol 4-dehydrogenase (EC 1.1.1.12) and for L-xylulose reductase (EC 1.1.1.10), and the amino acid sequences have recently been in WO 02/066616, which is incorporated herein by reference.

The L-xylulose reductase (EC 1.1.1.10) disclosed, e.g. in WO 02/066616, converts xylitol and NADP+ to L-xylulose and NADPH. The present invention provides an alternative L-xylulose reductase that is NADH dependent and can advantageously be used in place of the known NADPH dependent reductase.

A fungus as S. cerevisiae that is unable to utilise L-arabinose, but is a good ethanol producer, can be transformed with genes for aldose reductase, L-arabinitol 4-dehydrogenase, the present L-xylulose reductase, D-xylulose reductase and xylulokinase, it becomes capable to utilise efficiently L-arabinose and D-xylose. In such a strain the most abundant hexose and pentose sugars can be fermented to ethanol.

Sometimes organisms contain genes that are not expressed under conditions that are useful in biotechnological applications. For example, although it was once generally believed that S. cerevisiae cannot utilise xylose and it was therefore expected that S. cerevisiae did not contain genes encoding enzymes that would enable it to use xylose it has nevertheless been shown that S. cerevisiae does contain such genes (Richard et al.: “Evidence that the gene YLR070c of Saccharomyces cerevisiae encodes a xylitol dehydrogenase” in FEBS Lett, 457, 1999, 135-8). However, these genes are not usually expressed adequately. Thus, another aspect of our invention is to identify a gene for an L-xylulose reductase, which is NADH dependent, in a host organism itself and to cause the gene to be expressed in that same organism under conditions that are convenient for a biotechnological process, such as ethanolic fermentation of L-arabinose-containing biomass. We disclose a method of identifying a candidate for such a normally unexpressed gene, which is to search for similarity to SEQ ID NO 1. A candidate gene can then be cloned in an expression vector and expressed in a suitable host and cell-free extracts of the host tested for appropriate catalytic activity as described in Examples. When the normally unexpressed or inadequately expressed gene has been confirmed to encode the desired enzyme, the gene can then be cloned back into the original organism but with a new promoter that causes the gene to be expressed under appropriate biotechnological conditions. This can also be achieved by genetically engineering the promoter of the gene in the intact organism.

In yet another aspect of the invention the gene encoding L-xylulose reductase from a fungus, including fungi such as filamentous fungi that can have the ability to utilise L-arabinose, can now be easily identified by similarity to SEQ ID NO 1. This gene can then be modified for example by changing their promoters to stronger promoters or promoters with different properties so as to enhance the organism's ability to utilise L-arabinose.

A fungus may not naturally have the enzymes needed for lactic acid production, or it may produce lactic acid inefficiently. In these cases expression of the gene encoding lactate dehydrogenase (LDH) enzyme can be increased or improved in the fungus, and a fungus can then produce lactic acid more efficiently (e.g. WO 99/14335). Similarly, using methods known in the art, a fungus modified to use arabinose more efficiently as described in this invention can be further modified to produce lactic acid. As well as ethanol, lactate and sugar alcohols such as arabinitol and xylitol, other useful products can be obtained from the L-arabinose-utilizing fungi of the present invention. These fungi convert L-arabinose via the arabinose pathway to xylulose-5-phosphate, which is an intermediate of the pentose phosphate pathway. Thus, derivatives of the pentose phosphate pathway, such as aromatic amino acids, can also be produced as well as other substances derived from pyruvate, the common precursor of lactate and ethanol.

The transformed fungus is then used to ferment a carbon source such as biomass comprising agricultural or forestry products and waste products containing e.g. L-arabinose and possibly also other pentoses or other fermentable sugars. The preparation of the carbon source for fermentation and the fermentation conditions can be the same as those that would be used to ferment the same carbon source using the host fungus. However, the transformed fungus according to the invention consumes more L-arabinose than does the host fungus and produces a higher yield of ethanol on total carbohydrate than does the host fungus. It is well known that fermentation conditions, including preparation of carbon source, addition of co-substrates and other nutrients, and fermentation temperature, agitation, gas supply, nitrogen supply, pH control, amount of fermenting organism added, can be optimised according to the nature of the raw material being fermented and the fermenting microorganism. Therefore the improved performance of the transformed fungus compared to the host fungus can be further improved by optimising the fermentation conditions according to well-established process engineering procedures.

Use of a transformed fungus according to the invention to produce ethanol from carbon sources containing L-arabinose and other fermentable sugars has several industrial advantages. These include a higher yield of ethanol per ton of carbon source and a higher concentration of ethanol in the fermented material, both of which contribute to lowering the costs of producing, for example, distilled ethanol for use as fuel. Further, the pollution load in waste materials from the fermentation is lowered because the L-arabinose content is lowered, so creating a cleaner process.

Lignocellulosic raw materials are very abundant in nature and offer both renewable and cheap carbohydrate sources for microbial processing. Arabinose-containing raw materials are e.g. various pectins and hemicellulosics (such as xylans), which contain mixtures of hexoses and pentoses (xylose, arabinose). Useful raw materials include by-products from paper and pulp industry such as spent liquor and wood hydrolysates, and agricultural by-products such as sugar bagasse, corn cobs, corn fibre, oat, wheat, barley and rice hulls and straw and hydrolysates thereof. Also arabinane or galacturonic acid containing polymeric materials can be utilised.

Accordingly, the present invention enables advantageous means for the expression of the enzymes of the pathways, e.g. L-arabinose and, optionally, pentose phosphate pathway, for L-arabinose utilisation in microorganisms, especially in fungi.

### EXAMPLES

### Example 1

### Screening for Improved Growth on L-arabinose

The Saccharomyces cerevisiae strain H2651 (Richard et al.: “The missing link in the fungal L-arabinose catabolic pathway, identification of the L-xylulose reductase gene” in Biochemistry, 41, 2002, 6432-7) was used to screen an Ambrosiozyma monospora cDNA library for improved growth on L-arabinose. The H2651 contained all the genes of the fungal L-arabinose pathway. The Pichia stipitis XYL1 and XYL2 genes, coding for an aldose reductase and xylitol dehydrogenase respectively, were integrated into the URA3 locus. The strain expresses also the endogenous XKS1 gene coding for xylulokinase. The lad1 and lxr1 genes coding for the L-arabinitol dehydrogenase and the L-xylulose reductase from Hypocrea jecorina (Trichoderma reesei) were in separate multi-copy expression vectors with the LEU2 and URA3 marker genes.

Construction of the Ambrosiozyma monospora cDNA Library

The yeast Ambrosiozyma monospora (NRRL Y-1484) was cultivated in YNB medium (Difco) with 2% L-arabinose as the carbon source. The cells were grown overnight at 30° C. and harvested by centrifugation. Total RNA was extracted from the cells with the Trizol reagent kit (Life Technologies Inc.) according to the manufacturer's instructions. The mRNA was isolated from the total RNA with the Oligotex mRNA kit (Qiagen). The cDNA was synthesized by the SuperScript cDNA synthesis kit (Invitrogen) and the fractions containing cDNA were pooled and ligated to the SalI-NotI cut pEXP-AD502 vector (Invitrogen). The ligation mixture was transformed to the E. coli DH5 strain by electroporation in a ‘Gene pulser/micro pulser cuvette’ (BioRad) following the manufacturer's instructions. After overnight incubation about 30 000 independent colonies were pooled from ampicillin plates and stored in −80° C. in 50% glycerol +0.9% NaCl. Before extracting plasmids from the transformants the library was amplified by growing it for 4 hours in LB medium.

Screening the cDNA Library in S. cerevisiae

The S. cerevisiae strain H2651 was transformed with the cDNA library using the Gietz Lab Transformation Kit (Molecular Research Reagents Inc.). The transformants were plated on selective medium, lacking uracil, leucine and tryptophan, with 2% glucose as carbon source. After 2 days the plates were replicated on plates containing 1% L-arabinose as the carbon source. From the first colonies that appeared, plasmids were rescued and transformed to the E. coli strain DH5. The colonies that carried a plasmid from the library were identified by PCR with specific primers for the pEXP-AD502 vector f2: 5′-TATAACGCGTTTGGAATCACT-3′ and r: 5′-TAAATTTCTGGCAAGGTAGAC-3′. Plasmids were extracted and sequenced with the same primers.

One of the clones contained a plasmid that carried an open reading frame coding for a protein with 272 amino acids and a molecular mass of 29 495 Da. The deduced protein sequence had high homology to D-arabinitol dehydrogenases found from P. stipitis, Candida albicans and Candida tropicalis. In addition it had lower homology to the lxr1 gene product of H. jecorina that codes for L-xylulose reductase. The gene was named ALX1 for A. monospora L-xylulose reductase. The sequence is given in SEQ ID NO 1.

### Example 2

### Expression of the L-xylulose Reductase in S. cerevisiae

The ALX1 gene was isolated after SalI-NotI digestion and ligated to a multi-copy expression vector with uracil selection and PGK1 promoter. The expression vector was derived from the pFL60 by introducing SalI and NotI restriction sites to the multiple cloning site. The resulting plasmid was called p2178. It was then transformed to the S. cerevisiae strain CEN.PK2. This strain was called H2986 and was deposited with the deposition number DSM 15821 as described above.

Enzymatic Measurements in a Cell Extract

Cell extract from the strain H2986 was used to test the enzymatic activity for various substrates. Cells were cultivated overnight on selective glucose medium and cell extract was prepared with Y-PER reagent (Pierce). 0.5 ml of the reagent was used to lyse 0.1 g cells. Before the lysis ‘Complete protease inhibitors without EDTA’ (Roche) was added to the cell suspension.

The enzymatic activity with D-arabitol and xylitol was measured in a reagent containing 100 mM Tris-HCl, 0.5 mM MgC12 and 2 mM NAD+ or 2 mM NADP+. To start the reaction 100 mM sugar alcohol (final concentration) was added. All determinations were made in Cobas Mira automated analyser (Roche) at 30° C.

Activity was observed with sugar alcohols and NAD+ as substrate when the sugar alcohols were D-arabinitol or xylitol. The activities with these polyols were similar. As a control a similar strain was used that was only lacking the ALX 1. The control strain showed no activity. With the strain expressing the ALX1 no activity was observed with the C5 sugar alcohol L-arabinitol and the C6 sugar alcohols D-mannitol and D-sorbitol.

Purification of the His Tagged NAD-LXR1

A histidine-tag containing 6 histidines was added to the N-terminus of the protein by amplifying the gene by PCR using the following primers, 5′-GACTGGATCCATCATGCATCATCATCATCATCATATGACTGACTACATTC CAAC-3′ and 5′-ATGCGGATCCCTATATATACCGGAAAATCGAC-3′. Both primers have BamHI sites to facilitate cloning. The gene was cloned into the yeast multi-copy expression vector YEplac195 with PGK1 promoter (Verho et al.: “Identification of the first fungal NADP-GAPDH from Kluyveromyces lactis” in Biochemistry, 41, 2002, 13833-8). The resulting plasmid was named p2250. The gene was expressed in S. cerevisiae strain CEN.PK2 and the activity of the His-tagged protein was confirmed with enzyme activity measurements in a cell extract. For the purification of the protein the yeast strain expressing the histidine-tagged construct was grown overnight in 500 ml selective medium with 2% glucose and cells were collected. The cells were lysed with Y-PER reagent as described above and the lysate was applied into a NiNTA column (Qiagen).

Enzymatic Measurements with the Purified and Histidine Tagged Protein

Similar to the observations with the crude cell extract, activity was observed with sugar alcohols and NAD⁺ as substrate when the sugar alcohols were D-arabinitol or xylitol. No activity was observed with the C5 sugar alcohols L-arabinitol and adonitol (ribitol) and the C6 sugar alcohol dulcitol (galactitol). To start the reaction 100 mM sugar alcohol (final concentration) was added for all other sugar alcohols except dulcitol (galactitol). For dulcitol a final concentration of 10 mM was used. No activity was found when NAD⁺ was replaced by NADP⁺. The purified protein was also used to measure the reaction in the forward direction. The activity measurements in the forward direction with the sugar as a substrate were done in a reagent containing 100 mM Hepes-NaOH pH 7, 2 mM MgCl₂ and 0.2 mM NADH. A final concentration of 50 mM sugar was used to start the reaction for all other sugars except for D-sorbose. For D-sorbose a final concentration of 10 mM was used. In the direction with sugar and NADH as substrates activity was observed with L-xylulose and D-ribulose. A significantly decreased activity was observed with the pentulose sugar D-xylulose and no activity with the hexulose sugars D-sorbose, L-sorbose, D-psicose and D-fructose.

The purified protein was also used to determine the Michaelis Menten constants of the enzyme. The Kₘ for D-ribulose was 2,2±0,8 mM and the Kₘ for L-xylulose was 8,1±0,7 mM. The Vₘₐₓ values were 1900±330 nkat/mg for D-ribulose and 4100±100 nkat/mg for L-xylulose. The kinetic parameters for xylitol were 7,6±1,3 mM and 220±15 nkat/mg and for D-arabitol 2,4±0,1 mM and 210±11 nkat/mg.

Product Identification by HPLC

The purified enzyme was also used to identify the reaction products. For the forward direction a mixture of 100 mM Hepes-NaOH pH 7, 2 mM MgCl₂, 2 mM NADH, 2 mM pentulose was used. The products of the reverse reactions were identified in a reagent that contained 100 mM Tris-HCl, pH 9, 2 mM MgCl₂, 10 mM NAD⁺ and 20 mM polyol. 6 nkat of enzyme was added to the reagent and incubated for 3 hours at room temperature.

The products were identified with HPLC analysis. An Aminex Pb column (Bio-Rad) at 85° C. was used with water at a flow rate 0.6 ml/min. The polyols and pentuloses were detected with a Waters 410 RI detector.

Since the main activities were observed with D-ribulose and L-xylulose in the reducing reaction and with xylitol and D-arabinitol in oxidizing reaction, the products of these reactions were identified by HPLC. From L-xylulose xylitol was formed. The analysis allowed excluding that any arabinitol or adonitol (ribitol) was formed. From D-ribulose arabinitol was formed. The HPLC method that was used does not allow distinguishing between L- and D-arabinitol. In the reverse direction ribulose and xylulose was formed from D-arabinitol and xylulose was formed from xylitol. Also here the method does not allow distinguishing between L- and D-xylulose or L- and D-ribulose.

1. An isolated DNA molecule, characterised in that it comprises a gene encoding an enzyme protein which has an NADH dependent L-xylulose reductase activity.

2. An isolated DNA molecule according to claim 1, characterised in that the enzyme protein has a catalytic activity for the reversible conversion of a sugar which bears a keto group at carbon 2 (C2 position), to a sugar alcohol bearing a hydroxyl group at C2 in L-configuration in a Fischer projection.

3. An isolated DNA molecule according to claim 1, characterised in that the enzyme protein comprises an amino acid sequence of SEQ ID No. 2 or a functionally equivalent derivative thereof.

4. An isolated DNA molecule according to claim 1, characterised in that the enzyme protein is NADH dependent L-xylulose reductase of fungal origin.

5. An isolated DNA molecule according to claim 1, characterised in that said fungal origin is Ambrosiozyma monospora.

6. An isolated DNA molecule according to claim 1, characterised in that the gene comprises a nucleic acid sequence of SEQ ID No. 1 or a functionally equivalent derivative thereof.

7. An isolated DNA molecule according to claim 1, characterised in that the NADH dependent L-xylulose reductase exhibits a catalytic activity for reversible conversion of xylulose to xylitol.

8. A vector comprising the DNA molecule according to claim 1.

9. A genetically modified microorganism transformed with the DNA molecule according to claim 1 for expressing said NADH dependent L-xylulose.

10. A genetically modified microorganism according to claim 9, characterised in that it has an ability to utilise a sugar or a sugar alcohol.

11. A genetically modified microorganism according to claim 10, characterised in that it has an ability to utilise L-arabinose.

12. A genetically modified microorganism according to claim 9, characterised in that the microorganism produces derivatives of at least one of fungal L-arabinose pathway or of pentose phosphate pathway.

13. A genetically modified microorganism according to claim 9, characterised in that the microorganism contains at least genes of a fungal L-arabinose pathway, which encode enzymes of aldose reductase and of L-arabinitol 4-dehydrogenase, for expression thereof.

14. A genetically modified microorganism according to claim 13, characterised in that the microorganism contains genes of the fungal L-arabinose pathway, which encode enzymes of at least one of D-xylulose reductase or xylulokinase.

15. The microorganism of claim 14 further including genes encoding of D-xylulose of pentose phosphate pathway.

16. A genetically modified microorganism according to claim 9, characterised in that the microorganism produces at least one of arabinitol, xylitol, ethanol or lactic acid.

17. A genetically modified microorganism according to claim 9, characterised in that the genetically modified microorganism is a fungus.

18. The microorganism of claim 17 wherein the fungus is a yeast or a filamentous fungus.

19. A genetically modified microorganism according to claim 18, characterised in that the yeast is a strain of Saccharomyces species, Schizosaccharomyces species, Kluyveromyces species, Pichia species, Candida species or Pachysolen species.

20. A genetically modified microorganism according to claim 19, characterised in that the strain is S. cerevisiae.

21. A genetically modified microorganism according to claim 18, characterised in that the filamentous fungus is strain of Aspergillus species, Trichoderma species, Neurospora species, Fusarium species, Penicillium species, Humicola species, Tolypocladium geodes, Trichoderma reesei (Hypocrea jecorina), Mucor species, Trichoderma longibrachiatum, Aspergillus nidulans, Aspergillus niger or Aspergillus awamori.

22. A method for producing a fermentation product from a carbon source comprising a carbohydrate, characterised in that the method includes steps of culturing a genetically modified microorganism according to claim 9 in presence of a carbon source under fermentation conditions.

23. A method according to claim 22, characterised in that the carbon source comprises L-arabinose.

24. A method according to claim 22, characterised in that the carbon source comprises L-arabinose and the fermentation product is selected from a product of a fungal L-arabinose pathway and a product of a pentose phosphate pathway.

25. An enzyme protein which has an NADH dependent L-xylulose reductase activity and comprises an amino acid sequence encoded by a gene of a DNA molecule of claim 1.

26. An enzyme protein according to claim 25, characterised in that the enzyme protein comprises an amino acid sequence of SEQ ID No. 2 or a functionally equivalent derivative thereof.

27. An in vitro enzymatic preparation for producing conversion products from a carbon source, characterised in that said preparation comprises an enzyme protein which comprises an amino acid sequence encoded by DNA molecule according to claim 1.

28. A method of conversion of a sugar comprising contacting the sugar with an NADH dependent L-xylulose reductase enzyme, comprising an amino acid sequence encoded by a gene of a DNA molecule of claim 1, wherein the sugar has a keto group at C2 position and is converted to a sugar alcohol with a hydroxyl group at C2 in L-configuration in a Fischer projection, or for reversed conversion thereof.

29. The method of claim 28, characterised in that the enzyme is produced by a genetically engineered microorganism in a fermentation medium which comprises the sugar or the sugar alcohol, in fermentation conditions that enable conversion by said enzyme.

30. The method of claim 28, characterised in that the conversion is an in vitro enzymatic conversion.
